(19) United States
(12) Patent Application Publication
(10) Pub. No.: US 2026/0240887 A1
(43) Pub. Date: Aug. 20, 2026

(54) NUTRITIONAL INTERVENTIONS FOR OPTIMIZED ACUTE CONCUSSION CARE

(71) Applicant: Robert Baric, Cary, NC (US)

(72) Inventor: Robert Baric, Cary, NC (US)

(21) Appl. No.: 19/057,082

(22) Filed: Feb. 19, 2025

(51) Int. Cl. A61K 31/7068; A61K 31/121; A61K 31/191; A61K 31/197; A61K 31/198; A61K 31/202; A61K 31/205; A61K 31/375; A61K 31/57; A61K 31/593; A61K 33/06

(52) U.S. Cl. CPC A61K 31/7068; A61K 31/121; A61K 31/191; A61K 31/197; A61K 31/198; A61K 31/202; A61K 31/205; A61K 31/375; A61K 31/57; A61K 31/593; A61K 33/06

(57) ABSTRACT

Current concussion management protocols often miss a crucial intervention period immediately following injury. Metabolic intervention within the first 0-48 hours is critical to prevent far-reaching symptoms and ensure the brain's normal mechanisms function properly. We propose administration of a nutraceutical formulation comprising Vitamin C, Vitamin D, Omega-3 or DHA and/or EPA, creatine, N-acetyl cysteine, ketone esters, magnesium threonate, CDP-choline, curcumin, acetyl L-carnitine, pregnenolone, and pharmaceutically acceptable carriers thereof, up to three times per day to minimize damage caused by a potential concussion.

### INTRODUCTION

Every year, up to 60 million people suffer from traumatic brain injuries, including concussions, leading to staggering numbers: 27.16 million new cases, 49 million living with ongoing trauma, and a devastating 7 million years lost to disability globally (Amlerova et al., 2024). Shockingly, only 1% of all suspected concussions are reported (American Brain Foundation, 2023). The vast majority of reported concussions occur outside of sports, with falls, assaults, and motor vehicle incidents combined responsible for the majority, accounting for more than 60% of cases (American Brain Foundation, 2023). Children and the elderly have the highest incidence of these concussions unrelated to athletics. In contrast, sports-related concussions make up only a small fraction, around 14% (DePadilla et al., 2018). Alarmingly, despite the visibility of concussions related to athletics, as many as half of all sports-related concussions go unreported, highlighting a major gap in addressing head injuries in athletics. The consequences of these unreported and unaddressed concussions are dire on both an individual and global scale. According to a comprehensive Canadian study that followed 285 concussion patients using international sport concussion criteria, fewer than one-third of individuals with concussions fully recovered without intervention (Hiploylee et al. 2017). Additionally, those who experienced symptoms for more than three years showed no signs of recovery.

It is clear in current research that dysfunction post-concussion is not limited to the brain; rather, concussions have a profound impact on multiple body systems, often leading to long-term health issues (Mucha et al. 2019). These chronic sequelae cause severe long-term impacts, including cognitive impairments affecting academic/life performance, cardiac dysregulation, gastrointestinal dysfunction, hormonal dysregulation, decreased performance capacity in sports and other activities, mood changes including anxiety and depression often leading to interpersonal conflict, and neurodegenerative disease including dementia, Alzheimer's and encephalopathy (Lozano et al., 2017; Manley et al., 2017). Each concussion adds to the damage from previous ones, making the impact worse over time (Bailes et al., 2014). This might explain why even a mild head injury can lead to long-term adverse effects, especially if there have been previous untreated or asymptomatic traumas. This research suggests that a critical window within the first 0-48 hours post-injury exists for targeted metabolic intervention; further, immediate post-injury metabolic support is crucial for the prevention of extensive damage and lessening the time to recovery.

Failing to optimize support during this critical window may result in an energy crisis within the brain, followed by neuroinflammation and glymphatic dysfunction, leading to the inability to clear toxins and damaged tissue from the brain. While sports concussions are highly visible, it is essential to recognize that concussions can happen to anyone, anywhere.

### BACKGROUND

The Current Protocols: Critical Intervention Window Missed Our understanding of the importance of the critical window of the first 0-48 hours continues to expand. In addition to the commonly known metabolic responses to physical trauma, an entire secondary energetic crisis often occurs. It is well accepted that the brain is in a vulnerable state immediately post-concussion due to a state of energy deficit; the brain is temporarily unable to use its most readily available energy source: glucose. Immediately following a brain injury, glucose transport into the cell often becomes impaired, requiring a shift to ketones as an alternative energy source. If ketones are not available, the brain is unable to perform the essential functions of maintaining membrane potentials, removing toxins, and quelling inflammation.

Inflammation escalates in the brain post-injury as a normal response to tissue damage (Kalra et al., 2022). In normal circumstances, the body has mechanisms to resolve inflammation, including producing anti-inflammatory cytokines and mobilizing key immune cells, both dependent upon optimal Omega 3 fatty acid levels and an adequate energy source. With glucose utilization impaired post-brain injury, the mechanisms that prevent and resolve inflammation are impaired. Further, cells typically involved in neuronal protection, such as astrocytes and microglia, shift to create pro-inflammatory cytokine mediators such as IL-6, IL-17, TNF-α, and IFN-γ, creating a vicious cycle of escalating neuroinflammation (Lozano et al., 2017; Erta et al., 2012; Kalra et al., 2022). Short-term consequences include the high likelihood of secondary injury with “neural priming” setting the stage for more severe damage with a second head injury. Over time, this may result in chronic neurodegeneration, dementia, Alzheimer's, and encephalopathy (Acosta et al., 2015; Uryu et al., 2007; Tajiri et al., 2013). Application of current brain science suggests that targeted nutrients to quiet inflammation may be helpful in optimizing brain recovery post-injury (Chapek et al., 2020).

Immediately post-concussion, damage to the blood-brain barrier and cellular structures allows an influx of toxins and inflammatory proteins, including damaged tissue, into the brain. Researchers at the Nedergaard Lab at the University of Rochester Medical Center have delineated that post-TBI:
“Cell membranes and blood vessels rupture, spilling cytotoxic and inflammatory agents into the extracellular space and disrupting the blood supply, while blood-brain barrier breakdown leads to the influx of unfiltered blood-born constituents, shutting down regular cellular metabolism.” (Nedergaard et al., n.d.)

The brain's ability to detoxify requires an effective glymphatic system to clear waste and toxins during sleep, which is essential for recovery (Komaroff et al., 2021; Ferrara et al., 2022). In the absence of an accessible energy source, detoxification systems falter, allowing toxins and damaged tissue to remain in the brain, further augmenting the inflammatory response (Reddy et al. 2020). In addition, compromise of blood-brain barrier integrity, seen frequently with brain injury, allows toxins to be excluded from entry in normal circumstances and easy access into the brain (Shen et al., 2016). The combination of escalating inflammation and toxin buildup creates the perfect storm, promoting further damage and slowing recovery. Absent attention to these metabolic needs, effective rehabilitation becomes infeasible.

Additionally, the lack of an accessible energy source impairs the mechanisms that prevent and resolve inflammation, including the production of anti-inflammatory cytokines. Cells typically involved in neuronal protection, such as astrocytes and microglia, shift to create pro-inflammatory cytokine mediators, furthering neuronal damage (Kalra et al. 2022).

### CURRENT RECOVERY STRATEGIES

The cornerstone of recovery recommendations has centered on “brain rest” for many decades. According to the National Federation of State High School Associations (NFHS) and the Sports Medicine Advisory Committee (SMAC), the number one priority for concussion recovery is simple: rest (NFHS, 2002). Their suggested guide for concussion management in sports claims that “The first step in recovering from a concussion is rest. Rest is essential to help the brain heal.” Sources like Johns Hopkins Medicine and the University of Michigan Health & Medicine publicly recommend and concur that full rest is preeminent for concussion treatment and recovery. “In the first one to two days after suffering a concussion, near complete rest is important.” (Concussion Treatment & Recovery, University of Michigan Health, n.d.). Despite the emphasis on rest as the primary method for concussion recovery by these prominent organizations, this critical window immediately following a concussion remains an opportunity where metabolic intervention could prevent the worsening of long-term symptoms. Intervention within the first 0-48 hours, when the brain is most vulnerable to secondary damage due to energy deficits, is crucial, with timely metabolic support key to stabilization and healing (Chin et al. 2015; Leddy et al., 2018; Romanov et al., 2021).

Both cognitive and physical rehabilitation have now also become cornerstones of concussion recovery recommendations. The CDC submitted a report to Congress entitled Traumatic Brain Injury in the United States: Epidemiology and Rehabilitation, writing that TBI rehabilitation involves cognitive and physical therapies (Centers for Disease Control and Prevention, 2015). Cognitive rehabilitation addresses cognitive deficits (learning, comprehension) and behavior, while physical rehab focuses on improving mobility and ocular movement coordination. However, this approach also overlooks the critical metabolic aspects of brain recovery.

While the focus on cognitive and physical rehabilitation remains essential, it is crucial also to consider the brain's damaged mechanisms and the need for metabolic support to guide these processes back to balance and optimal function. The keys to a return to balance are the restoration of accessible energy for the brain, the resolution of inflammation, and the effective removal of toxins.

### THE CONSEQUENCES OF MISSED INTERVENTIONS

Each year, about 1.4 million US citizens visit emergency rooms for traumatic brain injuries (Lozano et al., 2017a). Despite the most current protocols, about 50% of people with TBI will experience further decline in their daily lives or die within 5 years of their injury (Centers for Disease Control and Prevention. n.d.). Research published in the Journal of Head Trauma Rehabilitation demonstrated that at 10 years post-injury, the most common co-morbidities developing post-brain injury, in order, were back pain, depression, hypertension, anxiety, fractures, elevated cholesterol, sleep disorders, panic attacks, osteoarthritis, and diabetes (Lozano et al., 2017). These disruptions can exacerbate symptoms and hinder recovery, illustrating the far-reaching consequences of brain injuries. These consequences involve multiple body systems, including gastrointestinal, musculoskeletal, visual, cardiac, cognitive-behavioral, hormonal, circadian rhythms disruption, including sleep, and the integrity of the blood-brain barrier. System Effects

### GASTROINTESTINAL

Numerous studies demonstrate a shift in gastrointestinal function following a traumatic brain injury (TBI). These shifts are felt both due to altered composition of the intestinal microbiome (dysbiosis) as well as increased permeability of the intestinal barrier, also known as “leaky gut.” A 2023 study out of Loyola University showed that individuals after TBI have increased levels of the potentially pathogenic bacteria Bilophila wadsworthia and decreased levels of bile acids in their feces and plasma (Cannon et al., 2022).

These changes can foster maldigestion, intestinal dysfunction, and neuroinflammation, disrupting the balance of the bidirectional gut-brain axis. This has central nervous system (CNS) implications as at least 80% of serotonin is created in the intestinal tract; thus, neurotransmitter production is disrupted, impairing CNS homeostasis (Cannon et al., 2023). Further, escalating inflammation can worsen CNS injury with antiinflammatory pathways impaired post-TBI (Hanscom et al., 2021). In addition to shifts in the microbiome, increased intestinal permeability can disrupt the gut-brain axis after TBI, as demonstrated first in studies of concussed rats (Hang et al., 2003).

Studies of the significance of intestinal permeability following TBI in humans are growing in number. A 2024 study in the International Journal of Molecular Science studied military “breachers” exposed to repetitive low-level blasts, many with symptoms of mild traumatic brain injury. Biomarkers of intestinal permeability were assessed pre-, post, and the next day after the blast and were found to be elevated (XVARC, 2024). Prior to the above study, human clinical data on mucosal barrier function following TBI was limited to studies of ICU patients. A 1998 study in the Journal of Trauma assessed gut permeability using lactulose/mannitol absorption ratios in TBI patients and detected impaired barrier function 4 days after injury but not earlier; permeability was correlated with disease severity and long-term prognosis (Faries et al., 1998).

There is general consensus based on multiple studies that intestinal permeability has a clear correlation with multiple forms of autoimmunity, with growing evidence of links to psychiatric disorders as well as neurodegenerative diseases, including Parkinson's disease and Alzheimer's. Thus, attention to repairing intestinal barrier integrity after TBI is vital to preventing these long-term chronic diseases.

### Visual

Traumatic brain injury (TBI) can result in a variety of visual issues affecting different parts of the optic system (Richman et al., 2016). Oculomotor behavior is typically categorized into convergence issues, fixations, smooth pursuits, and saccades (Hunfalvay et al., 2019). TBI, regardless of severity, is often associated with changes in all four categories, possibly resulting in significant impairment. This impairment translates to issues with response inhibition, short-term spatial memory, motor-sequence programming, visuospatial processing, and visual attention (Heitger et al., 2009). Literature trends suggest that impaired eye movement function correlates more strongly with increased post-concussive symptom loads and difficulties in daily living activities (Armstrong et al., 2018). This can manifest as dizziness with movement and car sickness.

### Cardiac

Traumatic brain injury causes a surge in catecholamines, which are hormones and neurotransmitters that regulate the fight-or-flight reaction, affecting cardiac cell receptors and leading to a systemic inflammatory response (Christensen et al., 2020; Coppalini et al., 2024). This response creates cellular dysfunction, which damages the brain and peripheral organs, including the heart.

Among the complications of TBI, cardiac injury is a frequent occurrence, affecting approximately 25-35% of patients with TBI (Coppalini et al., 2024). The most common heart problems in people with TBI are a higher risk for developing chronic cardiovascular disease and irregular heart rhythms. These issues occur up to 5 to 10 times more often in individuals with TBI history than in the general adult population (Izzy et al., 2023).

### Musculoskeletal

A recent meta-analysis has highlighted the significant increase in the risk of subsequent injuries following an initial concussion. Specifically, individuals who have sustained a concussion have a 2.5 times greater risk of experiencing a subsequent musculoskeletal injury or a second concussion (Smulligan et al., 2022). This heightened risk is consistent across different levels of competition, including professional, collegiate, and recreational athletes, as confirmed by another comprehensive review. Notably, the increased risk of injury remains present for up to 3 years following a concussion, well beyond the typical timeframe for concussion symptom resolution (Smulligan et al., 2022).

Recent studies continue to provide valuable insight into how traumatic brain injury (TBI) can significantly disrupt molecular pathways, leading to changes in bone formation and remodeling possibly contributed to by parathyroid disruption. Traumatic Brain Injury (TBI) has been linked to the abnormal development of bone tissue and increased callus formation (Bajwa et al., 2018). These abnormalities are primarily due to enhanced vascularization and the overactivation of systemic factors. Additionally, TBI can disrupt endocrine factors and neuropeptides, which may adversely affect bone health and metabolism.

### Blood-Brain Barrier

When a TBI is sustained, there is an immediate and direct impact on the blood-brain barrier, which may lead to detrimental long-term effects. Blood-brain barrier dysfunction, including activation of aquaporin-4 water channels, allows toxic materials usually excluded to enter the brain, such as bacterial lipopolysaccharide, environmental toxins, immune cells, and serum proteins, exacerbating the inflammatory response already set in motion by tissue damage. The compromise of the blood-brain barrier acts as the catalyst in the inflammatory cascade post injury (Cash et al., 2020; Xiong et al., 2018).

### Cognitive-Behavioral

Cognitive effects post-concussion are frequent, with a cross-sectional study following over 8,000 high school and college athletes finding the likelihood of developing Attention Deficit Hyperactivity Disorder (ADHD) or a Learning Disability (LD) were 2.93 and 2.0 times higher in those with a history of concussions (Nelson et al., 2016).

Individuals who have sustained a traumatic brain injury (TBI), regardless of the injury's severity, are also more prone to developing chronic mood disorders and psychiatric conditions. These can include anger, depression, and anxiety, as well as post-traumatic stress disorder (PTSD), due only in part to the psychological trauma associated with the incident, with neurotransmitter disruption and brain inflammation both acting as exacerbating factors. Depression and anxiety were noted in up to 30% of individuals as long-term developments post-injury (Jorge et al., 2004). This is due not only in part to the psychological trauma associated with the incident, but also with neurotransmitter disruption and brain inflammation both acting as compounding factors.

Studies have indicated that hippocampal damage is a significant indicator of mood disorders in patients assessed after a TBI. This damage leads to both short-term and long-term deficits in working, declarative, and episodic memory. Individuals with moderate to severe head injuries were found to have considerably smaller hippocampal volumes compared to those with mild TBI. Additionally, patients who developed mood disorders exhibited notably reduced hippocampal volumes (Jorge et al., 2007).

A comprehensive study on the cognitive effects of TBI demonstrated that individuals with a history of TBI performed worse on tasks involving executive function, attention, and memory than control groups (Brenner et al., 2023). This research highlighted that cognitive impairments are prevalent among TBI survivors, affecting their daily functioning and quality of life.

Suicide, specifically the time-to-suicide, was thoroughly studied among 860,892 soldiers, including 108,785 who had experienced at least one documented TBI. The increase in mental health diagnoses from before to after TBI was significantly greater compared to those without a TBI history (Brenner et al., 2023). Additionally, the time-to-suicide was 16.7% faster for soldiers with a TBI history than for those without.

Following a traumatic brain injury, anger and the ability to empathize are frequently reported concerns. From irritability to outbursts, up to one-third of patients report having negative behavioral symptoms following a concussion. Compared to the control participants, those with TBI reported a decreased ability to empathize emotionally, often associated with anger and low motivation (de Sousa et al., 2012).

These cognitive and emotional challenges also have a significant impact on caregivers. Caregivers of individuals with TBI often experience high levels of stress and burden, as they must manage not only the physical needs of the patient but also the psychological and behavioral changes (Verhaeghe et al., 2005). The demands of caregiving can lead to feelings of burnout, anxiety, and depression among caregivers. Research has shown that caregivers of TBI patients report lower quality of life and higher levels of psychological distress compared to caregivers of individuals with other chronic conditions (Kreutzer et al., 2009).

### Hormonal

TBI is associated with hormonal disruption on multiple levels (Bollerslev et al., 2013). A 2024 review in Brain and Spine highlighted several post-TBI neuroendocrine studies, identifying cortisol as the most frequently elevated hormone, peaking within 24 hours of injury (Magyar-Sumegi et al., 2024). Heightened levels of cortisol can lead to a maladaptive inflammatory response, perpetuating dysfunction of both the blood-brain barrier and neuronal cells, leading to a plethora of negative symptoms (Barton et al., 2021). After the initial cortisol spike, long-term cortisol dysregulation amongst other adrenal hormones is followed. Adrenal dysfunction is associated with tiredness, inadequate stress response, decreased memory, social issues, and mood or emotional disorders (Mahajan et al., 2023).

In addition to the disrupted adrenal axis, a broad range of pituitary hormone deficiencies are noted in one-third of adults and children post-TBI (Mahajan et al., 2023). As the pituitary gland produces hormones that stimulate the gonads to produce estrogen, progesterone, and testosterone, it is not surprising that studies confirm deficiencies in these reproductive hormones post-TBI (Lopez-Rodriguez et al., 2016). In 36 to 100% of males with severe TBI, investigation of luteinizing hormone (LH) and testosterone levels has identified a high incidence of low testosterone levels. Pituitary dysfunction following a traumatic brain injury, which can include conditions like hypopituitarism, hypothyroidism, and hypogonadism, can impact executive function, concentration, problem-solving ability, memory, and speech (Gillis-Januszewska et al., 2020).

### Sleep

Fatigue, insomnia, attentional deficits, and impaired cognitive function are symptoms of dysregulated sleep cycles following a traumatic brain injury, significantly reducing quality of life (Gottesman et al., 2024). Affecting 30-70% of individuals, these symptoms can be the result of various types of head trauma, with symptom expression impacted by the location and severity of the injury. Brain injury is often associated with disruption in natural circadian rhythms which function to regulate sleep/wake cycles via neuroendocrine hormonal pathways. This can manifest as either insomnia or hypersomnia.

Traumatic brain injury patients with sleep disturbances have been found to have longer inpatient hospital stays, higher costs of rehabilitation, and higher rates of functional disability (Lim et al., 2013). Sleep disturbance post-brain injury can also be impacted by pain elsewhere in the body, medications, and depression, which is much more common in persons with traumatic brain injury than in the general population (Jorge et al., 2004).

### DETAILED DESCRIPTION

### Current Approaches: Progress and Gaps in Concussion Care

The landscape of traumatic brain injury (TBI) research is rapidly expanding, shedding light on the intricate nature of its diagnosis and understanding of its far-reaching systemic effects. There is a surge in the development of assessment protocols, as well as effective musculoskeletal, visual, and cognitive rehabilitation protocols. However, a significant gap exists in current multifaceted rehabilitation approaches in addressing acute prevention of the myriad metabolic impacts leading to far-reaching systemic consequences. Amidst this evolving understanding, new methodologies are being explored to enhance assessment comprehensiveness. These include qEEG biomarkers or electrical signals recorded from the brain that can provide valuable insights into brain function and dysfunction. qEEG biomarkers have shown promise in assessing the severity and prognosis of traumatic brain injury (TBI) (Nishimura et al., 2022). Research indicates that quantitative EEG (QEEG) can identify, confirm, measure, and localize brain injury (Nishimura et al., 2022). These biomarkers help monitor cognitive function and detect abnormalities over time. In addition, various blood tests are being investigated as possible TBI biomarkers.

Eye-movement tracking devices are becoming more standardized and better understood for their value in assessing the state of the visual and autonomic nervous system (Alhilo et al., 2024). These advanced assessment methods offer a more detailed and holistic view of traumatic brain injuries. By using objective measurements, standardized procedures, and multi-faceted evaluation tools, clinicians can more accurately determine the severity and progression of concussions. This comprehensive approach helps understand the long-term effects of brain injuries and supports the development of more effective treatment and rehabilitation plans.

In addition to optimizing assessment, ongoing research continues to inform and update treatment protocols. At the 6th International Conference on Concussion in Sport in Amsterdam in 2022, guidelines were updated with recommendations and protocols to standardize the assessment and management of concussions (Broglio et al., 2024). These guidelines may cover various aspects of care, including initial evaluation, symptom monitoring, return-to-play criteria, and long-term management strategies. By providing a structured framework for assessment and treatment, the Amsterdam guidelines help ensure consistency and quality of care across different healthcare settings.

The 2024 National Athletic Trainers Association (NATA) protocols for concussion management mark a significant step forward by promoting a more comprehensive approach to care (Broglio et al., 2024). They now recommend early light exercise to enhance recovery, moving away from complete rest based on new evidence that physical activity aids brain healing. These protocols adopt a biopsychosocial model, integrating physical, psychological, and social factors, thus ensuring personalized treatment strategies that address mental health and social support needs.

Educational initiatives have been strengthened, aiming to equip athletes, coaches, parents, and school administrators with thorough concussion knowledge to improve prevention, recognition, and management. The protocols also emphasize multidimensional assessments, using cognitive tests, pre-competition qEEGs, and visual/vestibular screenings for tailored treatment plans.

Despite advancements in assessment, the treatment landscape for TBIs remains relatively sparse, with only a few methods available to address the complex acute and chronic treatment needs of individuals affected by concussions. Efforts to develop a simplified, comprehensive approach are underway, driven by the growing understanding of the multifaceted nature of concussions and their impact on overall health and well-being.

One notable treatment approach is Vizstim, which promotes cognitive rehabilitation through a series of exercises designed to engage both the musculoskeletal and visual systems. By targeting these specific areas, Vizstim aims to improve cognitive efficiency over the long term (VIZSTIM, n.d.).

Various sources are now recommending targeted nutritional support for the brain in the acute TBI phase (Carneiro et al., 2022). Currently, however, there is no systems-wide approach to address the magnitude of the brain's unique metabolic demands during this vulnerable period. Much progress remain s to be made in the realm of acute metabolic system failures to bridge this gap and optimize outcomes.

### A Comprehensive Solution

In response to the growing need for enhanced concussion care, a scientifically formulated nutritional solution appears to be the most viable option to support individuals immediately and in the acute phase after a suspected head injury. This innovative approach emphasizes prompt intervention, optimizing brain energy, and consuming essential nutrients to foster recovery. By addressing these critical needs, easy-to-use nutritional support aims to minimize the extent of damage, accelerate the healing process, and reduce the incidence of secondary complications.

Unlike conventional methods that primarily address symptom management, this strategy takes a proactive stance by leveraging the brain's inherent mechanisms for healing and recovery in acute settings. Supported by robust research and expert insights, this solution offers an effective and integrative method for managing concussions, bridging the gaps in current treatment, and promoting overall well-being.

Through a carefully curated blend of nutrients, vitamins, minerals, antioxidants, and neuroprotective compounds, each ingredient is specifically selected to provide the brain's various known system failure points with an optimized energy source to utilize, mitigate neuroinflammation, and improve glymphatic detoxification.

### Mitigating Neuroinflammation

### Vitamin D

Vitamin D is not only a vitamin but functions as a hormone in many body processes (Sassi et al., 2018; Scrimgeour et al., 2021). It has broad anti-inflammatory, increased cardiovascular ejection volume, and neuroprotective effects, acting as an “immunomodulator” to dampen excessive immune responses. Vitamin D also helps to keep calcium in balance, which is important in TBI as excess calcium released in response to cellular damage fosters neuronal cell death (Sassi et al., 2018; Lawrence et al., 2016).

### Omega-3

Omega-3 essential fatty acids have been shown to decrease the damage caused by brain injury (Thau-Zuchman et al., 2019; Wu et al. , 2023; Finnegan et al., 2022; Patch et al., 2021; Scrimgeour et al., 2021a). They are essential components of cell membranes, protecting cells from oxidative damage. The fatty acids docosahexaenoic acid (DHA) and eicosapentaenoic acid (EPA) are both neuroprotective via multiple mechanisms, including increasing BDNF, a brain-derived neurotrophic factor (Ziaei et al., 2023). They are also essential in resolving inflammation (Norling et al., 2010).

### N-Acetyl Cysteine (NAC)

NAC, as a precursor to Glutathione, has broadly active antioxidant and inflammatory properties. It also aids the liver in detoxifying the body from potentially harmful toxins and chemicals. In a 2013 study of 81 military personnel suffering combat-related/artillery-blast head injuries, oral NAC was used within 24 hours of injury (Hoffer et al., 2013). The percentage of those experiencing complete resolution of their symptoms more than doubled in the NAC group. Topical glutathione reduced cell death in an experimental model of TBI in mice (Roth et al., 2014).

### Curcumin

Curcumin, an active ingredient in turmeric, has been a well-known anti-inflammatory for centuries. In a mouse model of brain injury, curcumin was shown to decrease brain swelling following TBI and reduce interleukin-1, an inflammatory chemical released in the brain (Laird et al., 2010). It also decreased the activation of aquaporin-4, a water channel protein in cell membranes, including the blood-brain barrier. When activated, it allows toxins to flow through to the usually well-protected brain. A 2022 meta-analysis revealed that curcumin, the active form in turmeric, exhibited protective effects on TBI via modulation of multiple cell signaling pathways (Khayatan et al., 2022). Curcumin exhibits beneficial immunomodulatory functions and protective capacities in multiple different TBI models (Garodia et al., 2023; Zhang et al., 2023).

### Vitamin C

Further brain injury, known as “secondary injury,” is felt to be due to several mechanisms, including oxidative stress. Vitamin C becomes quickly depleted during times of critical illness, allowing the damage cascade to continue. Vitamin C is a powerful antioxidant that has been shown to improve outcomes in patients with acute TBI (Shen et al., 2016; Razmkon et al., 2011).

### Pregnenolone

Pregnenolone is a neuroprotective and neuroactive inhibitory steroid that helps the nervous system to calm down. It has been found to be low in patients with a history of TBI. In a randomized study of 30 military veterans with mild TBI, pregnenolone has been shown to help with insomnia, irritability, and hypervigilance (all symptoms that overlap with post-concussive syndrome) (Marx et al., 2016).

### Optimizing the Brain's Energy Consumption

### Ketones

Ketone bodies, produced in the liver from fatty acids, are the most efficient fuel source for the brain. Rapidly after TBI, an “energetic crisis” occurs as glucose metabolism in the brain is altered. This is followed by neuroinflammation at the site of injury that further impairs metabolism and energy production in the brain. Ketone bodies are naturally created in the fasting state. Studies in animals show that fasting after moderate TBI causes an increase in ketones, which are associated with neuroprotection, improved mitochondrial function, decreased oxidative stress, and maintenance of cognitive function. Exogenous ketones can provide the brain with much-needed energy in the early hours to days after concussion. A 2024 review in Current Opinion in Clinical Nutrition and Metabolic Care highlights the opportunity for incorporating ketones in a comprehensive TBI management approach (Arora et al., 2024).

Acetyl-L carnitine

Acetyl-L-carnitine assists the mitochondria in the production of energy. Acetyl-L-carnitine exerts neuroprotection via anti-inflammatory effects as well as its effects on the regulation of neuronal synaptic plasticity by counteracting post-trauma excitotoxicity (Hiskens et al., 2023; Tashakori-Miyanroudi et al., 2022). In several animal studies, it has been shown to reduce damage from TBI. In a 2010 study using a rat model of TBI, treatment with acetyl-L-carnitine during the first 24 hours after TBI improved behavioral outcomes and reduced brain lesion volume in immature rats within the first 7 days after injury (Chapek et al., 2020; Scafidi et al., 2010). A 2023 study, also using a rat TBI model, demonstrated that acetyl-L-carnitine treatment showed protective effects against those observed in the untreated rat TBI group (Hiskens et al., 2023).

### Citicoline

Citicoline, or CDP-choline, is a component of human metabolism as an intermediate in the synthesis of phosphatidylcholine, the main phospholipid in cellular membranes, including neuronal and glial cells. It is also a factor in the balance of the acetylcholine neurotransmitter system necessary to carry messages from the brain to the rest of the body. Citicoline has been shown to increase the incorporation and metabolism of glucose, as well as decrease lactate levels in the brain during ischemia (Secades et al., 2021; Watanabe et al., 1975). Choline has shown promise in improving outcomes in patients post-TBI (Meshkini et al., 2016; Secades et al., 2021; Watanabe et al., 1975; Krishna et al., 2012). A 2017 meta-analysis of patients with head injury treated with citicoline within the first 24 hours showed increased independence in the follow-up period (Meshkini et al., 2016). A retrospective matched-pair analysis from Austria in 2018 demonstrated reduced rates of intensive care unit (ICU) mortality, in-hospital mortality, and 6-month mortality, as well as unfavorable outcomes (Trimmel et al., 2018).

While the largest human prospective trial of choline in TBI, the Citicoline Brain Injury Treatment Trial (COBRIT), failed to show a significant difference, there were however methodologic issues with the study. COBRIT was a double-blind randomized and placebo-controlled trial of 1296 patients with mild, moderate, or severe TBI (Zafonte et al., 2009). The inclusion of mild, moderate, and severe TBI may have limited the power of the study to detect significant differences in these populations separately. In addition, the sample size was not large enough to prove an optimal odds ratio, and compliance of only 44.4% of patients taking more than 75% of the medication further limited the study design (Zafonte et al., 2009). Further study is indicated.

### Additional Support

### Creatine

Creatine has shown promise for attenuating symptoms of concussion, mild TBI, and depression (Forbes et al., 2002; Roschel et al., 2021; Newman et al., 2023). A 2023 review of 15 articles support the benefit of creatine in patients post-TBI (Newman et al., 2023). Of note, the Results are only clinically significant after a month of supplementation. Creatine showed efficacy as a neuroprotective agent in battling the chronic manifestations that lead to oxidative stress and cognitive function post-brain injury (Newman et al., 2023).

### CONCLUSION

Despite huge advances in understanding the pathophysiology of concussion and TBI, which should lead to better assessment of potential damage and more therapeutic strategies, there remains a gap in understanding how to adequately address the metabolic needs of the critical 24-48 hour window following concussion. Our targeted nutraceutical formulations comprising the above-described nutrients provide one solution to synergistically maintain a metabolic energy source following concussion, further facilitating cerebral detoxification and reducing intercellular swelling/ inflammation. Additionally, this formulation can be taken prophylactically.

1-52.

53. A method for reducing damage caused by head trauma, or for minimizing potential brain damage caused in a brain-damaging event before the fact, comprising administration of the pharmaceutical composition of claim 1, either before the brain-damaging event commences, or one to three times daily after head trauma is suspected.

54-59.
